(19) United States
(12) Patent Application Publication
(10) Pub. No.: US 2026/0263494 A1
(43) Pub. Date: Sep. 10, 2026

(54) Dual-Binding Tandem Peptide-Conjugated Listeriolysin O (LLO) Or Doxorubicin (DOX) For Targeted Cancer Cell Delivery, Reducing Resistance Development, Minimizing Off-Target Toxicity, And Enhancing Efficacy By Synergistic Effect In Directly Inducing Cancer Cell Apoptosis, Boosting Anti-Tumor Immune Response And Converting a Cold Tumor Microenvironment Into A Hot One

(71) Applicant: Xiangfei Cheng, Montgomery Village, MD (US)

(72) Inventor: Xiangfei Cheng, Montgomery Village, MD (US)

(21) Appl. No.: 19/074,994

(22) Filed: Mar. 10, 2025

(51) Int. Cl. A61K 31/704; A61K 38/16; A61K 47/64; C07K 14/435

(52) U.S. Cl. CPC A61K 31/704; A61K 38/164; A61K 47/64; C07K 14/435; C07K 2319/50

(57) ABSTRACT

The present innovative dual-binding tandem peptide-cytotoxin conjugates target various cancers. The conjugate strategy incorporates reducing off-target toxicity by combining the targeted delivery and linker which is specifically cleaved by cathepsin B highly expressed in cancer cells, decreasing resistance through specifically dual-binding to cancer cells and targeting multiple pathways including directly killing cancer cells through toxin, DOX or LLO, and anti-tumor immune action, and enhancing efficacy by synergistic effect through directly killing cancer cells by the toxin, and upregulating anti-tumor immune response via blocking PD-1 and PD-L1 interaction by anti-PD-L1 peptide and converting a cold tumor microenvironment into a hot one. Leveraging this strategy, I designed two types of conjugates: tandem peptide-conjugated DOX and tandem peptide-conjugated LLO, each utilizing a distinct class of cytotoxic agent. Upon the targeted delivery into cancer cells, the conjugate is cleaved at the linker by cathepsin B, releasing targeting peptide, anti-PD-L1 peptide and the toxin.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO A SEQUENCE LISTING, A LARGE TABLE, OR A COMPUTER PROGRAM LISTING APPENDIX ON READ-ONLY OPTICAL DISC

Not Applicable

### BACKGROUND OF THE INVENTION

Cancer therapy is often limited by the development of off-target toxicity, drug resistance and target downregulation, which significantly reduce treatment efficacy and lead to disease relapse. Conventional conjugates rely on target recognition and cytotoxic agents to eliminate cancer cells; however, tumor cells frequently develop resistance through mechanisms such as downregulation of target, upregulation of drug efflux pumps, activation of alternative survival pathways, genetic mutations, and modification of the tumor microenvironment. These resistance mechanisms allow cancer cells to evade drug-induced cell death, necessitating alternative therapeutic strategies.

Target downregulation presents one of major challenges, particularly in targeted therapies. Tumor cells can evade immune surveillance or targeted drug action by reducing the expression of surface antigens, receptors, or signaling molecules that serve as critical binding sites for therapeutic agents. This phenomenon severely limits the efficacy of monoclonal antibodies, antibody-drug conjugates (ADCs), and targeted small-molecule inhibitors.

The tumor microenvironment (TME) plays a crucial role in determining the response to immunotherapy. Cold tumors-those that lack immune cell infiltration and have an immunosuppressive environment-are often resistant to immune checkpoint inhibitors and other immunotherapies. By exerting pro-and anti-tumorigenic actions, tumor-infiltrating immune cells can profoundly influence tumor progression and the success of anti-cancer therapies. Conversion of a cold tumor environment into a hot one face challenges and is critical for immunotherapy.

Despite recent advancements, there remains an urgent need for novel therapeutic strategies that simultaneously overcome drug resistance, off-target toxicity and target downregulation. In my present innovation, I integrated targeted delivery, chemical therapy, immunotherapy, promoting converting a cold tumor microenvironment into a hot one, and reducing resistance development within a single molecule. I innovated two types of tandem peptide-conjugated conjugates: Tandem peptide-conjugated Doxorubicin (DOX) and Tandem peptide-conjugated bacterial Listeriolysin O (LLO). The innovative conjugates are created by conjugating tandem peptide to DOX or LLO. These innovative conjugates integrate the targeted delivery and reducing resistance development by dual-binding to cancer cells, linker being specifically targeted by cathepsin B highly expressed in cancer cells, and synergistic role in killing cancers via inhibiting PD-L1 by a component of the conjugate, anti-PD-L1 peptide, converting a cold tumor microenvironment into a hot one, and directly inducing cancer cell death by toxic agents, DOX or LLO, leading to enhance efficacy, reduce the development of resistance, minimize side effects, and decrease dose. In my present innovation description, DOX and LLO are referred to as cytotoxic agents or toxins. The tandem peptide-conjugated DOX and tandem peptide-conjugated LLO are collectively referred to as dual-binding tandem peptide-cytotoxin conjugates.

### BRIEF SUMMARY OF THE INVENTION

The present invention introduces an innovative treatment strategy achieved through an innovative conjugate, tandem peptide-conjugated DOX or LLO, strategically designed to the directed delivery of the conjugates into target cells by dual binding to targets, not only directly induce cancer cell death by the toxin but also enhance immune response by inhibiting PD-L1, and triggering anti-tumor immune response, resulting in a comprehensive and effective strategy for intervention in multiple myeloma (MM), acute myeloid leukemia (AML), chronic lymphocytic leukemia (CLL), lymphomas, lung tumor, colorectal tumor, ovarian tumor, breast cancer, glioblastoma, pancreatic cancer, head and neck cancer, melanoma, epidermal squamous cell carcinoma, bladder cancer, prostate cancer, hepatocellular carcinoma and gastric cancer.

In my present invention, these innovative molecules are created by conjugating tandem peptide to DOX or LLO. The tandem peptide consists of transportan (TP), linker (FRRG), targeting peptide, linker, anti-PD-L1 peptide and linker from N-terminus to C-terminus (FIG. 1). Tandem peptide is conjugated to DOX or LLO through the linker at C-terminus of the tandem peptide. The targeting peptide is selected based on its binding specificity to different cancers. The linker, a peptide (FRRG), is specifically cleavable by cathepsin B in cancer cell cytoplasm. Cancer cells highly express cathepsin B compared to normal cells.

These inventions encompass the synthesis methodology, structural elucidation, and applications of the innovative molecules for the intervention in various cancers. They are designed for intervention in MM, AML, CLL, lymphomas, lung tumor, colorectal tumor, ovarian tumor, breast cancer, glioblastoma, pancreatic cancer, head and neck cancer, melanoma, epidermal squamous cell carcinoma, bladder cancer, prostate cancer, hepatocellular carcinoma and gastric cancer, providing a pathway for the development of novel anticancer treatment.

### Advantages of Tandem Peptide-Conjugated DOX and Tandem Peptide-Conjugated LLO

The Targeted Delivery, Enhance Efficacy and Reduce Toxicity: The targeted deliver by dual-binding to both targeting peptide's binding site and PD-L-1 and synergistic role in killing cancer cells by DOX or LLO, enhancing anti-tumor immune response by inhibiting PD-L1 through anti-PD-L1 peptide, while also converting a cold tumor microenvironment into a hot one through the immune action, targeting multiple pathways, enables lower doses, enhanced efficacy, minimized off-target toxicity, and reduced resistance development.

Comprehensive Pathway Blockade: Anti-PD-L1 peptide enhances anti-tumor immune response by inhibiting PD-L1 while DOX and LLO increase the immune response and enable a cold tumor microenvironment into a hot one through inducing immunogenic cell death and or activating macrophage to promote a pro-inflammatory phenotype. These synergistic roles enhance cancer cell killing more effectively.

Reduce Resistance Development: By concurrently binding two targets in cancers and synergistic roles in killing cancers cells, these innovative conjugates reduce the likelihood of cancer cells developing resistance, thereby prolonging treatment efficacy.

Reduce Off-target Toxicity: Cancer cells highly express cathepsin B compared to normal cells. The linkers are designed for specifically cleavable by cathepsin B. Both targeting peptide and anti-PD-L1 peptide, as components of these conjugate, direct the targeted delivery of the conjugates into cancer cells. This synergistic role reduces off-target toxicity.

### BRIEF DESCRIPTION OF THE SEVERAL VIEWS OF THE DRAWING

FIG. 1: Structure of Tandem Peptide-Conjugated Cytotoxin, DOX or LLO. The tandem peptide comprises transportan, linker, targeting peptide, linker, anti-PD-L1 peptide and Linker from N-terminus to C-terminus. Tandem peptide is conjugated to DOX or LLO through the linker at C-terminus of tandem peptide. The targeting peptide is selected based on its binding specificity to different cancers. All linker is same. The linker is a peptide, FRRG. The link is specifically cleavable by cathepsin B which is highly expressed in cancer cells compared to normal cells.

FIG. 2. The Targeted Delivery and Mechanism of Action of The Tandem Peptide-Conjugated DOX. a) The conjugate, Tandem Peptide-Conjugated DOX, is specifically delivered into cancer cells through specific dual-binding of tandem peptide to cancer cells in which targeting peptide specifically binds to cancer cell and anti-PD-L1 peptide specifically binds to PD-L1, and synergistic role with the intracellular trafficking peptide, transportan, a component of the conjugate, in crossing cell membrane; b) Following the targeted delivery of the conjugate into cancer cells, the linker in the conjugate is cleaved by cathepsin B which is highly expressed in cancer cells, leading to release of anti-PD-L1 peptide, DOX and targeting peptide in cytoplasm; c) Anti-PD-L1 peptide inhibit PD-L1. DOX damages DNA in nucleus, leading to cancer cell apoptosis; d). PD-1 and PD-L1 interaction is blocked by anti-PD-L1 peptide, leading to attenuating T cell exhaustion, thereby enhances anti-tumor immune response both in tumor microenvironment and systemic immune response, and DOX induces immunogenic cell death, which promotes high DAMP signals for DC maturation and cytotoxic T lymphocyte activation, resulting in an increased anti-tumor immune response in the tumor microenvironment and a heightened systemic immune response; e) Upregulate the anti-tumor immune response via blockage of PD-1 and PD-L1 interaction by anti-PD-L1 peptide, cytokines released by immune cells and signals from the tumor microenvironment; f) Immune cells infiltrate tumors, leading to converting a cold tumor microenvironment into a hot one and eliminating tumor.

FIG. 3. The Targeted Delivery and Mechanism of Action of The Tandem Peptide-Conjugated LLO. a) The conjugate, Tandem Peptide-Conjugated LLO, is specifically delivered into cancer cells through specific dual-binding of tandem peptide to cancer cells in which targeting peptide specifically binds to cancer cell and anti-PD-L1 peptide specifically binds to PD-L1 and synergistic role with the intracellular trafficking peptide, transportan, a component of the conjugate, in crossing cell membrane; b) Following the targeted delivery of the conjugate into cancer cells, the linker in the conjugate is cleaved by cathepsin B which is highly expressed in cancer cells, leading to release of anti-PD-L1 peptide, LLO and targeting peptide in cytoplasm; c) Anti-PD-L1 peptide inhibit PD-L1. LLO disrupts the phagosomal membrane; d) PD-1 and PD-L1 interaction is blocked by anti-PD-L1 peptide, leading to attenuating T cell exhaustion, thereby enhances the anti-tumor immune response both in the tumor microenvironment and systemically. The LLO disrupts phagolysosomal membranes that enhance antigen cross-presentation of tumor-derived peptides and activate cytosolic immune sensors, and boost macrophage activation to a pro-inflammatory phenotype, leading to enhancing anti-tumor immune response both in tumor microenvironment and systemic immune response; e) Upregulate the anti-tumor immune response via blockage of PD-1 and PD-L1 interaction by anti-PD-L1 peptide, cytokines released by immune cells and signals from the tumor microenvironment; f) Immune cells infiltrate tumors, leading to converting a cold tumor microenvironment into a hot one and eliminating tumor.

### DETAILED DESCRIPTION OF THE INVENTION

Cancer remains a leading cause of morbidity and mortality worldwide, despite significant advancements in targeted therapies, immunotherapies, and antibody-drug conjugates (ADCs). One of the greatest challenges in cancer treatment is overcoming therapeutic resistance, which arises due to downregulation of target, tumor heterogeneity, genetic mutations, and microenvironmental factors that enable cancer cells to evade therapy. Resistance mechanisms include target mutation and downregulation, drug efflux, metabolic alterations, and immune escape, ultimately leading to disease progression and treatment failure.

In addition to resistance, off-target toxicity presents a major barrier to effective cancer therapy. While targeted therapeutics aim to minimize damage to normal tissues, unintended interactions with non-malignant cells result in significant adverse effects. Antibody cross-reactivity, systemic toxicity of chemotherapeutics, and immune-related adverse events associated with checkpoint inhibitors all contribute to therapy limitations, reducing patient compliance and overall treatment success.

Another critical challenge is ensuring therapeutic efficacy across diverse tumor types. Tumor heterogeneity, limited drug penetration, and immune evasion mechanisms hinder the long-term success of both small-molecule inhibitors and biologics. To enhance treatment outcomes, novel strategies such as combination therapy, tumor-specific activation mechanisms, and biomarker-driven patient selection are being explored.

My present innovation is dedicated to addressing key challenges in resistance development, off-target toxicity, and efficacy limitations in cancer therapy, with a focus on innovative strategies to enhance therapeutic outcomes. By leveraging rational design and advanced conjugation technologies, the next generation of cancer therapeutics can achieve greater clinical success.

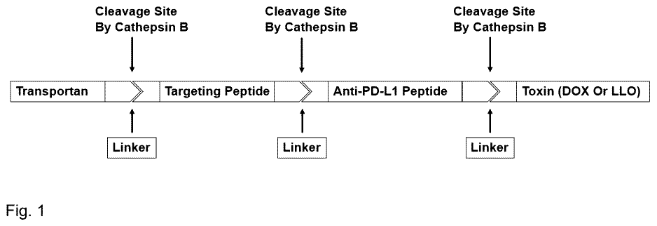

In my present invention, I invented two types of conjugates: Tandem peptide-conjugated DOX and Tandem peptide-conjugated LLO. Tandem peptide for both types' conjugates is same, which consists of transportan (GGWTLNSAGYLLGKINLKALAALAKKIL), linker (FRRG), targeting peptide, linker, anti-PD-L1 peptide (CVRARTR) and linker from N-terminus to C-terminus (FIG. 1). Transportan is an intracellular trafficking peptide. The targeting peptide is selected based on its binding specificity to different cancers. The linker links DOX or LLO to tandem peptide at C-terminus of tandem peptide. The linker is designed for cleavable by cathepsin B which highly expresses in cancer cells compared to normal cells, leading to reducing off-get toxicity risk. Normal cells inherently exhibit low cathepsin B expression (1-4). Moon et. al. (2022) developed a DOX-based conjugate. They reported that the DOX exhibits low toxicity toward normal cells and immune cells by their innately low cathepsin B expression (5). Notably, although my conjugate uses DOX as a toxin, my innovative conjugates are distinct from theirs. This will be discussed in detail in the discussion section of this patent description. To address challenge in resistance development via target evasion, my innovative conjugate is designed to possess two binding peptides, targeting peptide and anti-PD-L1 peptide, which can bind to targeting peptide's binding site and PD-L1 in cancer cells, respectively. Most of tumors highly express PD-L1. Additionally, anti-PD-L1 peptide not only plays role in the targeted delivery of conjugate but also inhibit PD-L1 to attenuate T cell exhaustion. The targeting peptide is selected based on its binding specificity to different cancers. The intracellular trafficking peptide, transportan, a component of the conjugates, facilitates conjugate across the cell membrane efficiently, enabling targeted intracellular delivery of the conjugates by playing a synergistic role with the dual-binding of targeting peptide and anti-PD-L1 peptide to their targets. Additionally, in the conjugates for intervention in glioblastoma, the transportan facilitates the blood-brain barrier (BBB) crossing through this synergistic mechanism. The tandem peptide-conjugated DOX or LLO, is designed for interventions in MM, AML, CLL, lymphomas, lung tumor, colorectal tumor, ovarian tumor, breast cancer, glioblastoma, pancreatic cancer, head and neck cancer, melanoma, epidermal squamous cell carcinoma, bladder cancer, prostate cancer, hepatocellular carcinoma and gastric cancer. Following the targeted delivery of the conjugate into cancer cells, anti-PD-L1 peptide, targeting peptide, toxin (DOX and LLO) are released by cleaving the linker through cathepsin B.

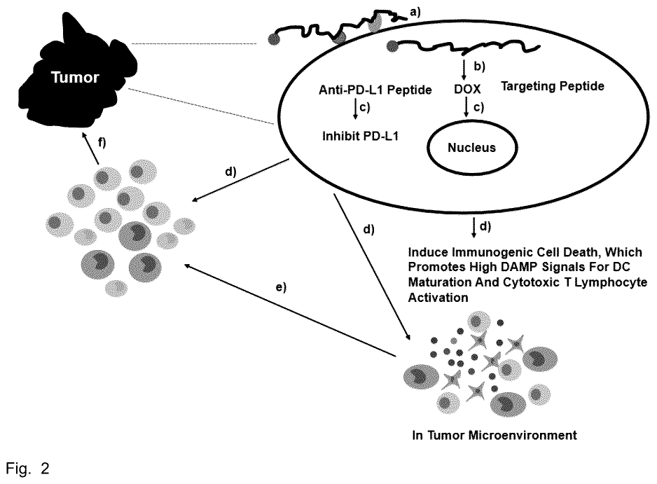

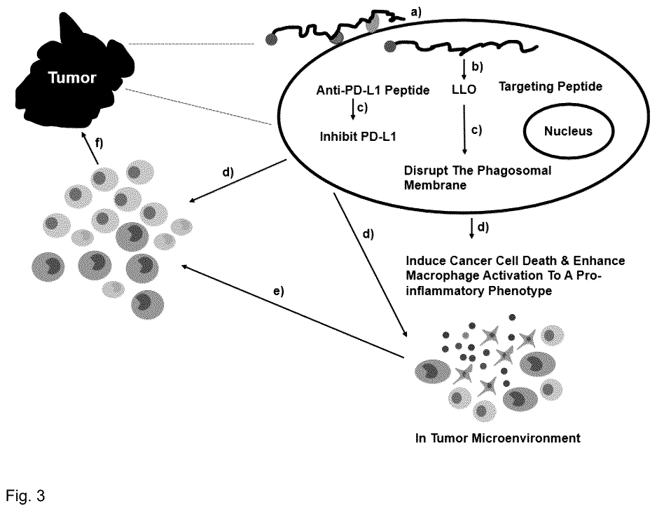

In these present innovations, the scientific rational (FIG. 2-3) behind these innovative conjugate, tandem peptide-conjugated DOX or LLO, for intervention in MM, AML, CLL, lymphomas, lung tumor, colorectal tumor, ovarian tumor, breast cancer, glioblastoma, pancreatic cancer, head and neck cancer, melanoma, epidermal squamous cell carcinoma, bladder cancer, prostate cancer, hepatocellular carcinoma and gastric cancer is that the conjugate, tandem peptide-conjugated DOX or tandem peptide-conjugated LLO, is specifically delivered into cancer cells through specific dual-binding of the targeting peptide and anti-PD-L1 peptide to their targets in cancer cells and synergistic role with the intracellular trafficking peptide, transportan, a component of the conjugate, in crossing cell membrane. Following the targeted delivery of tandem peptide-conjugated DOX or tandem peptide-conjugated LLO into cancer cells, anti-PD-L1 peptide, targeting peptide, DOX or LLO are released by cleaving linker through cathepsin B. The anti-PD-L1 peptide attenuates T cell exhaustion to enhance anti-tumor immune response by inhibiting PD-L1. The released DOX induce immunogenic cell death (ICD) in cancer cells, which promotes high DAMP signals for dendritic cells (DC) maturation and cytotoxic T lymphocyte activation (5). The released LLO disrupts phagolysosomal membranes that enhance antigen cross-presentation of tumor-derived peptides and activate cytosolic immune sensors, and boost macrophage activation to a pro-inflammatory phenotype (6). Taken together, these conjugates kill cancer cells by targeting multiple pathways, including directly inducing cancer cell death, immune action and converting a cold tumor microenvironment into a hot one. Schrank et al. (2025) developed CD47-LLO. They reported that the release of LLO from CD47-LLO is critical to enhance macrophage activation to a pro-inflammatory phenotype (6). In my present innovative conjugates, by engaging two targets, dual binding (binding of targeting peptide to cancer cells and binding of anti-PD-L1 peptide to PD-L1 in cancer cells) minimizes the likelihood of tumor escape mechanisms, enhancing treatment efficacy. Cathepsin B highly express in cancers cells. The linker is specifically cleavable by the enzyme. Taken together, both targeted delivery and the specifically cleavage of linker reduce off-target toxicity. The targeted delivery and synergistic effect of these groundbreaking molecules in directly killing cancer cells, enhancing anti-tumor immune response and converting a cold tumor microenvironment into a hot one drive a transformative strategy to overcome resistance, minimize off-target toxicity, reduce the required dose, and enhance efficacy.

### Method

Tandem peptide is synthesized and acylated. The tandem peptide-conjugated LLO is generated following the method described by Schrank et al (2025) (6). Briefly, the tandem peptide is modified with the chemistry reagent DBCO-PEG4-NHS ester for 30 min at room temperature. Excess cross-linker was removed by high performance liquid chromatography (HPLC). The buffer of LLO protein is exchanged for PBS buffer, pH 7.2 with a PD-10 column (GE Healthcare, 17085101), followed by modification with the chemistry cross-linking reagent SPDP-PEG11-azide. Excess reagent was removed by buffer exchange, and then the modified LLO protein was mixed with tandem peptide and allowed to react overnight at room temperature. Unconjugated tandem peptide and LLO proteins were removed by affinity and size exclusion chromatographic methods, respectively. The tandem peptide-conjugated DOX is generated following the method described by Moon et al (5). In brief, the tandem peptide-conjugated DOX is synthesized by one-step conjugation of N-terminal acylated tandem peptide and doxorubicin (DOX) via EDC/NHS coupling chemistry. First, Ac-tandem peptide, DOX, NHS and EDC are dissolved in anhydrous DMF and then DIPEA is subsequently added in mixture solution. After 6 h of incubation, prodrug is purified using HPLC and lyophilized.

Experimental adjustments and optimizations may be necessary to achieve the desired product efficiently and selectively. The conjugate synthesis can be performed using alternative methods to those described above.

### Anti-Pd-L1 Peptide, DOX and LLO Play Roles in Killing Cancer Cells

PD-L1 is expressed on several types of tumors, including urothelial cancers, gastrointestinal cancers, lung cancer, breast cancer, melanoma, and ovarian cancer, as well as on tumor infiltrating immune cells in the tumor microenvironment (7-13). Gnong et al. reported PD-L1 expression in cell lines elevated after radiation via PI3K/AKT and STAT3 pathways. Moreover, down regulation of PD-L1 could reduce radiation resistance through promoting apoptosis. Radiotherapy in combination with anti-PD-L1 antibody synergistically elevated antitumor immunity through promoting CD8 T cell infiltration and decreasing the accumulation of MDSCs and tumor infiltrating regulatory T cells (iTregs) in a mouse model (14). The FDA approval for pembrolizumab which blocks binding of PD-L1 to PD-1 in lung cancer was received in October 2014(15 ).

Tumor cells acquire different ways to evade the host immunity in the tumor microenvironment, called cancer immunosurveillance. Over the past decades, many studies of cancer immunosurveillance indicated that one of the superlative important components of the underlying mechanism is an immunosuppressive co-signal (immune checkpoint) mediated by PD-1/PD-L1 in the tumor microenvironment. Several preclinical reports also represented that blockage of the interaction between PD-1 and PD-L1 enhances the T-cell response and mediates antitumor activity (16-17). Anti-PD-1/PD-L1 therapy would make significant clinical benefits via promoting regression of advanced and metastatic tumors and improving survival. Importantly, anti-PD-1/PD-L1 therapy could have durable effects, tolerable toxicity, and is applicable to a wide spectrum type of cancers, especially in solid tumors (18, 17). Several PD-1 and PD-L1 inhibitors have been approved for cancer therapy by FDA (19).

DOX is most commonly used to treat cancers of the bladder, breast, stomach, lung, ovaries, thyroid, soft tissue sarcoma, multiple myeloma, and Hodgkin's lymphoma. The commonly used DOX-containing regimens may include Adriamycin, cyclophosphamide (AC), Taxotere, AC, Adriamycin, bleomycin, vinblastine, dacarbazine, bleomycin, etoposide, AC, vincristine, procarbazine, and prednisone, cyclophosphamide, Adriamycin, vincristine, prednisone and 5-fluorouracil, AC. Doxil is mainly used for the treatment of ovarian cancer where it has progressed or recurred after platinum-based chemotherapy, or for the treatment of AIDS-related Kaposi's sarcoma (20). The exact mechanism of action of DOX is complex and still unclear. DOX interacts with the DNA by intercalation thus, inhibiting macromolecular biosynthesis (21, 22). This further inhibits the progression of the enzyme topoisomerase II, and relaxes supercoils in DNA for transcription. DOX stabilizes the topoisomerase II complex after it has broken the DNA chain for replication, preventing the DNA double helix from being resealed and thereby stopping the process of replication. Another mechanism of DOX is its ability to generate free radicals that induce DNA and cell membrane damage (23).

LLO is a pH-dependent pore-forming toxin with high cytolytic activity in acidic chambers. Bergelt et al. reported that LLO functions as the cytotoxic component of an immunotoxin, directly inducing tumor cell death (24). The B3-LLO immunotoxin has been strategically designed to exploit pH-dependent activation: in a neutral environment, LLO remains in an oxidized, low-cytotoxic state. However, upon internalization into acidic endosomal compartments, LLO regains maximal activity, disrupting the phagosomal membrane and inducing tumor cell death (24). This innovative approach establishes a novel platform for cancer immunotherapy.

Advancements in targeted liposome technology have enhanced the delivery of chemotherapeutic agents, enabling their direct accumulation within tumors. These drugs, capable of penetrating tumor cell membranes, improve treatment efficacy (25). However, systemic circulation inevitably exposes normal cells to cytotoxic effects, leading to off-target toxicity (26). Additionally, while some highly cytotoxic agents remain membrane-impermeable, their therapeutic potential is significantly higher when released into the cytosol (27, 28). LLO offers a promising solution to these challenges. Kullberg et al. developed an immunoliposome incorporating both LLO and bleomycin, a potent cytotoxic agent, for targeted delivery to HER2-overexpressing breast cancer cells. Trastuzumab facilitated tumor targeting, while LLO mediated liposomal cargo to pass into the cytoplasm, enabling cytosolic release of bleomycin (29). This approach led to a remarkable 57,000-fold increase in cytotoxicity compared to free bleomycin, underscoring LLO's potential in improving therapeutic outcomes (29).

The use of DOX and LLO as toxins targeting cancer cells faces challenges related to toxicity and efficacy. To address this challenge, targeted delivery is imperative. In my present innovative conjugates, the DOX or LLO are conjugated to tandem peptide. Following the targeted delivery of conjugate into cancer cells, the linkers in conjugates are cleaved by cathepsin B to release anti-PD-L1, targeting peptide, DOX or LLO. They exert synergistic roles in killing cancer cells by directly inducing cancer cell apoptosis and enhancing anti-tumor immune response, converting a cold tumor microenvironment into a hot one.

### Targeting Peptides' Roles in the Targeted Delivery of the Innovative Conjugate into Cancer Cells

Cell penetrating peptides are unique, 5-30 amino acid long peptides that are able to breach cell membrane barriers and carry cargoes intracellularly in a functional form. Sri et al. (2023) reported that peptides exhibit lower affinity and a shorter half-life in the body than antibodies. Conversely, peptides demonstrate higher efficiency in tissue penetration and cell internalization than antibodies. Regardless of the pros and cons of peptides, they have been used as tumor-homing ligands for delivering carriers (such as nanoparticles, extracellular vesicles, and cells) and cargoes (such as cytotoxic peptides and radioisotopes) to tumors. Additionally, tumor-homing peptides have been conjugated with cargoes such as small-molecule or chemotherapeutic drugs via linkers to synthesize peptide-drug conjugates. In addition, peptides selectively bind to cell surface receptors and proteins, such as immune checkpoints, receptor kinases, and hormone receptors, subsequently blocking their biological activity or serving as hormone analogs. Furthermore, peptides internalized into cells bind to intracellular proteins and interfere with protein-protein interactions. Thus, peptides demonstrate great application potential as multifunctional players in cancer therapy. It was reported that peptides target cancers for the intervention in various cancers, including RGD4C (ACDCRGDCFCG) targeting Integrin av33 for the intervention in melanoma, colon tumor, ovarian tumor and glioblastoma (30-32), iRGD1 (CRGDR) and iRGD2 (KGPDC) targeting Integrin av33 for the intervention in Glioblastoma and melanoma, LyP-1 (CGQKRTRGC) targeting P32 for the intervention in Melanoma (33), K237 (HTMYYHHYQHHL) targeting VEGFR-2 and mUNO (CSPGAK) targeting CD206 for the intervention in breast tumor (34, 35), Octreotide (FCFWKTCT) targeting SSTR2/5, specific for the intervention in pancreatic and breast tumor (36), IL4RPep-1 (CRKRLDRNC) targeting IL4R for the intervention in lung tumor, breast tumor and colon tumor (37-41), P3 (NALDEYCEDKNR) targeting HLA-E for the intervention in multiple myeloma (42), EPI-X4 (LVRYTKKVPQVSTPTL) targeting CXCR4 for the intervention in AML cells (43), TV1 (TRICCGCYWNGSKDVCSQSCC) targeting ion channels for the intervention in hepatocellular carcinoma (44), Bld-1 (CSNRDARRC) targeting bladder cancer cells for intervention in bladder cancer (45), AAD (AADNAKTKSFPV) and GP-5 (IHKDKNAPSLVP) targeting gastric cancer cell for intervention in gastric cancer (46, 47), LN1 (TGTPARQ), H3 (GFIATLCTKVLDFGIDKLQLIEDK) and Prost1 (WQPDTAHHWATL) targeting LNCaP prostate cancer cells for intervention in prostate cancer (48, 49), GE11 (YHWYGYTPQNVI) targeting EGFR for intervention in triple negative breast cancer, lung tumor, head and neck tumor and squamous cell carcinoma of the skin (50-52), and blades B1B2-derived peptides, BB1B2 (FDAIAEIGNQLYLFKDGKYW), targeting CD44 for the intervention in CLL Cells (53). BIRD2 (NVYTEIKCNSLLPLDDIVRV) binds to the BH4 domain, blocking Bcl-2-IP3 R interaction and thus inducing Ca2+-mediated apoptosis in chronic lymphocytic leukemia, multiple myeloma, and follicular lymphoma cells, including cells resistant to ABT-263, ABT-199, or the Bruton's tyrosine kinase inhibitor Ibrutinib (54, 55).

McCandless et al. (2021) identified a synthetic, non-naturally occurring 12-amino acid long peptide that was termed cardiac targeting peptide (CTP: APWHLSSQYSRT) due to its ability to transduce cardiomyocytes in vivo. Studies looking into its mechanism of transduction identified two lung targeting peptides (LTPs), S7A (APWHLSAQYSRT) and R11A (APWHLSSQYSAT) (56). These peptides robustly transduced human bronchial epithelial cell lines in vitro and mouse lung tissue in vivo. This uptake occurred independently of clathrin mediated endocytosis. Biodistribution studies of R11A showed peak uptake at 15 minutes with uptake in liver but not kidneys, indicating primarily a hepatobiliary mode of excretion. Cyclic version of both peptides was ~100-fold more efficient in permeating cells than their linear counterparts. As proof of principle, they conjugated anti-spike and anti-envelope SARS-COV-2 siRNAs to cyclized R11A and demonstrate anti-viral efficacy in vitro. Their work presented two novel lung-specific cell penetrating peptides that could potentially deliver myriad therapeutic cargoes to lung tissue (56).

Kwon et al. engineered a neuron-targeting nanoparticle which can mediate intracellular trafficking of siRNA cargo and achieved silencing of mRNA and protein levels in cultured cells. They created transportan-RVG (TP-RVG) tandem peptide, and encapsulated the TP-RVG tandem peptide and siRNA in nanoparticle, leading to deliver the siRNA to CNS by passing through blood-brain barrier. They discovered that, when administered systemically into animals with brain injuries, neuron-targeted nanoparticles can accumulate into the tissue adjacent to the injured site. Their results demonstrated that RVG-targeted nanoparticle mediated knockdown of Caspase 3 mRNA analyzed by RT-qPCR and protein analyzed by western blotting (57). Townsenda et al. studied Tetanus toxin C fragment conjugated nanoparticles for targeted drug delivery to neurons. They developed nanoparticles from PLGA-PEG-biotin polymers and used biotin binding proteins (avidin, streptavidin, or neutravidin) as crosslinkers for protein conjugation. The tetanus toxin C fragment was modified and conjugated with nanoparticles, allowing targeted binding to neuroblastoma cells, while not targeting liver or endothelial cells (58).

In my present inventions, the targeting peptide, as a component of the innovative conjugates, plays roles in directed delivery of the conjugates into cancer cells.

### Discussion

Cancer treatment faces significant challenges, including drug resistance, target downregulation, off-target toxicity, and limited efficacy. In my present innovative conjugates, the strategy involves reducing off-target toxicity by combining the targeted delivery and incorporating linker which is specifically cleavable by cathepsin B that highly expresses in cancer cells for releasing anti-PD-L1 peptide, toxin and targeting peptide, decreasing resistance by specifically dual-binding to cancer cells and targeting multiple pathways including directly killing cancer cells by toxin, DOX or LLO, and anti-tumor immune action, and enhancing efficacy through leveraging synergistic roles in directly inducing cancer cells apoptosis by the toxin and enhancing anti-tumor immune response via blockage of PD-1 and PD-L1 interaction by anti-PD-L1 peptide and converting a cold tumor microenvironment into a hot one. Leveraging this strategy, I designed two types of conjugates: tandem peptide-conjugated DOX and tandem peptide-conjugated LLO, each utilizing a distinct class of cytotoxic agent. These conjugates retain the same targeting mechanism, employing an enzymatic cleavable linker to release the cytotoxic agent, anti-PD-L1 peptide and targeting peptide upon delivering into the target cancer cells.

Moon et al. (2022) developed anti-PD-L1 peptide-conjugated prodrug nanoparticles for targeted cancer immunotherapy. In their study, the functional peptide, consisted of anti-PD-L1 peptide and cathepsin B-specific cleavable peptide, is conjugated to a DOX, resulting in prodrug nanoparticles of PD-NPs via intermolecular interactions. They reported that the PD-NPs are taken up by PD-L1 receptor-mediated endocytosis and then induce ICD in cancer cells by DOX release. Concurrently, PD-L1 blockade by PD-NPs disrupt the immune-suppressing pathway of cancer cells, resulting in proliferation and reinvigoration of T lymphocytes. In tumor models, PD-NPs accumulate within tumor tissues via enhanced permeability and retention (EPR) effect and induce immune-responsive tumors by recruiting a large number of immune cells (5). Importantly, my innovative conjugates are distinguishable from theirs. Beyond anti-PD-L1 peptide, linker and DOX, my innovative conjugates possess additional targeting peptide and an intracellular traffic peptide, transportan. The targeting peptide and anti-PD-L1 peptide play synergistic roles with intracellular traffic peptide in the targeted delivery of the conjugates into cancer cell. Tumor cells frequently develop resistance through mechanisms such as downregulation of receptor and targets, activation of alternative survival pathways and genetic mutations to escape attacking. In my innovative conjugates, both targeting peptide and anti-PD-L1 peptide bind to cancer cells. This dual-binding strategy reduces the risk of escape mechanisms, leading to decreasing resistance development and a lower likelihood of cancer relapse. Notably, in the conjugate for intervention in glioblastoma, transportan enhances BBB penetration.

Schrank et al (2025) developed a CD 47 antibody-conjugated LLO for cancer immunotherapy. Using a microbial-inspired strategy, they engineered an antibody-toxin conjugate (ATC) that targets the ‘don't eat me’ signal CD47 linked to the bacterial toxin, LLO, from the intracellular bacterium Listeria monocytogenes via a cleavable linker (CD47-LLO). CD47-LLO promotes cancer cell phagocytosis by macrophages followed by LLO release and activation to form pores on phagolysosomal membranes that enhance antigen cross-presentation of tumor-derived peptides and activate cytosolic immune sensors. CD47-LLO treatment in vivo significantly inhibited the growth of both localized and metastatic breast and melanoma tumors and improved animal survival as a monotherapy or in combination with checkpoint blockade. Together, these results demonstrate that designing ATCs to promote immune recognition of tumor cells represents a promising therapeutic strategy for treating multiple cancer (6). Their conjugates utilize an antibody (Ab) as a binding ligand to specifically target cancer-associated antigens. However, in my present innovative conjugates, targeting peptide is as a binding ligand to bind cancer cells. Beyond the targeting peptide, my present innovative conjugates possess both additional anti-PD-L1 peptide and transportan. By leveraging dual-binding of targeting peptide and anti-PD-L1 peptide to cancer cells, this dual-binding strategy reduces the risk of escape mechanisms, leading to decreasing resistance development and a lower likelihood of cancer relapse. Anti-PD-L1 peptide not only plays roles in specifically binding to cancer cells but also enhance anti-tumor immune response and promoting converting a cold tumor microenvironment into a hot one by inhibiting PD-L1 to attenuate T cell exhaustion.

Notably, although individual components or mechanisms described herein may be supported by prior literature, my present invention is based on a novel and non-obvious integration of these components in a specific and synergistic manner that yields functional advantages not suggested or taught in the prior art. The invention described herein is not a mere aggregation of known components, but rather a purposeful integration of structural and functional elements designed to achieve a synergistic effect and a novel molecular construct with properties that reduce toxicity risk, enhance efficacy, and decrease resistance.

### Rationale for Designing Both DOX- and LLO-Based Conjugates

DOX induces immunogenic cell death in cancer by damaging DNA, which promotes high DAMP signals for dendritic cells (DC) maturation and cytotoxic T lymphocyte activation (5). LLO disrupts phagolysosomal membranes that enhance antigen cross-presentation of tumor-derived peptides and activate cytosolic immune sensors, and boost macrophage activation to a pro-inflammatory phenotype. Cancer cells often evade therapy through various mechanisms. Even though my innovative molecules incorporate strategies to reduce resistance development, the design of two types of conjugates-one utilizing the toxin DOX and the other utilizing the toxin LLO-increases the likelihood that at least one toxin remains effective, even if resistance arises against one of the toxins.

### In Conclusion

The dual-binding tandem peptide-conjugated DOX and tandem peptide-conjugated LLO were invented to enable targeted intervention in various malignancies. This innovation leverages targeting peptide and anti-PD-L1 precision targeting, attacking multiple pathways, including directly inducing cancer cell death by toxin, DOX or LLO, and enhancing anti-tumor immune response both in tumor microenvironment and systemically, while minimizing toxicity, reducing resistance development, and enhancing efficacy, making it a promising strategy for cancer therapy.

1: A Dual-binding tandem peptide-cytotoxin conjugates comprising:
a tandem peptide which can specifically bind to both targeting peptide's binding site and PD-L1 expressed on cancer cell membrane is conjugated to a cytotoxic agent via an enzymatic cleavable linker, wherein the linker is peptide, FRRG, specifically cleavable by cathepsin B;
wherein the tandem peptide comprises a transportan (GGWTLNSAGYLLGKINLKALAALAKKIL), a linker (FRRG), a targeting peptide, a linker (FRRG), an anti-PD-L1 peptide (CVRARTR) and a linker (FRRG) from N-terminus to C-terminus;
wherein the targeting peptide that specifically binds to a target expressed on the surface of cancer cells is selected from the group consisting of P3, EPI-X4, BB1B2, BIRD2, IL4RPep-1, S7A, R11A, RGD4C, K237, mUNO, iRGD1, iRGD2, Octreotide, Lyp-1, Prost1, GE11, TV1, ADD, GP-5, LN1, H3, Bld-1 and RVG1;
wherein the cytotoxic agent is selected from:
(a) DOX, a small molecule toxin that induce cancer cells death, or
(b) LLO, a protein toxin that induces cancer cells death;
wherein the tandem peptide is conjugated to DOX or LLO through a linker (FRRG) at C-terminus of the tandem peptide; wherein the linker is specifically cleavable by cathepsin B. Dependent Claims:

2: The tandem peptide-cytotoxin conjugate of claim 1, wherein the conjugate comprises the targeting peptide, P3 (NALDEYCEDKNR), specifically designed for multiple myeloma intervention, and wherein the tandem peptide is conjugated to either DOX or LLO, which are distinct cytotoxic agents linked via the linker (FRRG) at C-terminus of the tandem peptide.

3: The tandem peptide-cytotoxin conjugate of claim 1, wherein the conjugate comprises the targeting peptide, EPI-X4 (LVRYTKKVPQVSTPTL), specifically designed for acute myeloid leukemia (AML) intervention, and wherein the tandem peptide is conjugated to either DOX or LLO, which are distinct cytotoxic agents linked via the linker (FRRG) at C-terminus of the tandem peptide.

4: The tandem peptide-cytotoxin conjugate of claim 1, wherein the conjugate comprises the targeting peptide, BB1B2 (FDAIAEIGNQLYLFKDGKYW), specifically designed for lymphomas intervention, and wherein the tandem peptide is conjugated to either DOX or LLO, which are distinct cytotoxic agents linked via the linker (FRRG) at C-terminus of the tandem peptide.

5: The tandem peptide-cytotoxin conjugate of claim 1, wherein the conjugate comprises the targeting peptide, BIRD2 (NVYTEIKCNSLLPLDDIVRV), specifically designed for CLL, multiple myeloma, and follicular lymphoma intervention, and wherein the tandem peptide is conjugated to either DOX or LLO, which are distinct cytotoxic agents linked via the linker (FRRG) at C-terminus of the tandem peptide.

6: The tandem peptide-cytotoxin conjugate of claim 1, wherein the conjugate comprises the targeting peptide, IL4RPep-1 (CRKRLDRNC), specifically designed for lung tumor, breast tumor and colon tumor intervention, and wherein the tandem peptide is conjugated to either DOX or LLO, which are distinct cytotoxic agents linked via the linker (FRRG) at C-terminus of the tandem peptide.

7: The tandem peptide-cytotoxin conjugate of claim 1, wherein the conjugate comprises the targeting peptide, S7A (APWHLSAQYSRT) or R11A (APWHLSSQYSAT), specifically designed for lung tumor intervention, and wherein the tandem peptide is conjugated to either DOX or LLO, which are distinct cytotoxic agents linked via the linker (FRRG) at C-terminus of the tandem peptide.

8: The tandem peptide-cytotoxin conjugate of claim 1, wherein the conjugate comprises the targeting peptide, RGD4C (ACDCRGDCFCG), specifically designed for melanoma, colon tumor, ovarian tumor and glioblastoma intervention, and wherein the tandem peptide is conjugated to either DOX or LLO, which are distinct cytotoxic agents linked via the linker (FRRG) at C-terminus of the tandem peptide, with the exception that the tandem peptide-conjugated LLO does not apply to glioblastoma intervention due to LLO's inability to cross the blood-brain barrier.

9: The tandem peptide-cytotoxin conjugate of claim 1, wherein the conjugate comprises the targeting peptide, K237 (HTMYYHHYQHHL), specifically designed for breast cancer intervention, and wherein the tandem peptide is conjugated to either DOX or LLO, which are distinct cytotoxic agents linked via the linker (FRRG) at C-terminus of the tandem peptide.

10: The tandem peptide-cytotoxin conjugate of claim 1, wherein the conjugate comprises the targeting peptide, mUNO (CSPGAK), specifically designed for breast cancer intervention, and wherein the tandem peptide is conjugated to either DOX or LLO, which are distinct cytotoxic agents linked via the linker (FRRG) at C-terminus of the tandem peptide.

11: The tandem peptide-cytotoxin conjugate of claim 1, wherein the conjugate comprises the targeting peptide, iRGD1 (CRGDR) or iRGD2 (KGPDC), specifically designed for glioblastoma and melanoma intervention, and wherein the tandem peptide is conjugated to either DOX or LLO, which are distinct cytotoxic agents linked via the linker (FRRG) at C-terminus of the tandem peptide, with the exception that the tandem peptide-conjugated LLO does not apply to glioblastoma intervention due to LLO's inability to cross the blood-brain barrier.

12: The tandem peptide-cytotoxin conjugate of claim 1, wherein the conjugate comprises the targeting peptide, Octreotide (FCFWKTCT), specifically designed for pancreatic and breast tumor intervention, and wherein the tandem peptide is conjugated to either DOX or LLO, which are distinct cytotoxic agents linked via the linker (FRRG) at C-terminus of the tandem peptide.

13: The tandem peptide-cytotoxin conjugate of claim 1, wherein the conjugate comprises the targeting peptide, LyP-1 (CGQKRTRGC), specifically designed for melanoma intervention, and wherein the tandem peptide is conjugated to either DOX or LLO, which are distinct cytotoxic agents linked via the linker (FRRG) at C-terminus of the tandem peptide.

14: The tandem peptide-cytotoxin conjugate of claim 1, wherein the conjugate comprises the targeting peptide, LN1 (TGTPARQ), H3 (GFIATLCTKVLDFGIDKLQLIEDK) or prost1 (WQPDTAHHWATL), specifically designed for prostate intervention, and wherein the tandem peptide is conjugated to either DOX or LLO, which are distinct cytotoxic agents linked via the linker (FRRG) at C-terminus of the tandem peptide.

15: The tandem peptide-cytotoxin conjugate of claim 1, wherein the conjugate comprises the targeting peptide, GE11 (YHWYGYTPQNVI), specifically designed for epidermal squamous cell carcinoma, triple negative breast cancer, lung and head and neck tumor intervention, and wherein the tandem peptide is conjugated to either DOX or LLO, which are distinct cytotoxic agents linked via the linker (FRRG) at C-terminus of the tandem peptide.

16: The tandem peptide-cytotoxin conjugate of claim 1, wherein the conjugate comprises the targeting peptide, TV1 (TRICCGCYWNGSKDVCSQSCC), specifically designed for hepatocellular carcinoma intervention, and wherein the tandem peptide is conjugated to either DOX or LLO, which are distinct cytotoxic agents linked via the linker (FRRG) at C-terminus of the tandem peptide.

17: The tandem peptide-cytotoxin conjugate of claim 1, wherein the conjugate comprises the targeting peptide, ADD (AADNAKTKSFPV) or GP-5 (IHKDKNAPSLVP), specifically designed for gastric tumor intervention, and wherein the tandem peptide is conjugated to either DOX or LLO, which are distinct cytotoxic agents linked via the linker (FRRG) at C-terminus of the tandem peptide.

18: The tandem peptide-cytotoxin conjugate of claim 1, wherein the conjugate comprises the targeting peptide, Bld-1 (CSNRDARRC), specifically designed for bladder tumor intervention, and wherein the tandem peptide is conjugated to either DOX or LLO, which are distinct cytotoxic agents linked via the linker (FRRG) at C-terminus of the tandem peptide.

19: The tandem peptide-cytotoxin conjugate of claim 1, wherein the conjugate comprises the targeting peptide, RVG1 (YTIWMPENPRPGTPCDIFTNSRGKRASNG), specifically designed for glioblastoma intervention, and wherein the tandem peptide is conjugated to DOX, which is linked via the linker (FRRG) at C-terminus of the tandem peptide.

20: The molecule of claim 1, wherein a modified form thereof retains the core chemical structure and mechanism of action of the molecule, and wherein modifications, including variations in the targeting peptide, cytotoxic agent and linkers, are designed to enhance efficacy, reduce toxicity, improve bioavailability, or optimize pharmacokinetics, with such modifications including structural alterations, functional group substitutions, stereochemical modifications, or other chemically relevant changes that preserve or enhance the molecule's therapeutic or diagnostic properties while maintaining the overall mechanism of action.
